(19) United States
(12) Patent Application Publication
(10) Pub. No.: US 2010/0004521 A1
(43) Pub. Date: Jan. 7, 2010

(54) IMPLANTABLE VOLTAIC CELL

(76) Inventor: Spencer J.G. Epps, Rockland, DE (US)

Correspondence Address: BLANK ROME LLP, ONE LOGAN SQUARE, PHILADELPHIA, PA, 19103, US

(21) Appl. No.: 12/299,752

(22) PCT Filed: May 7, 2007
(86) PCT No.: PCT/US07/11144

§ 371 Date: Jul. 23, 2009

Related U.S. Application Data
(60) Provisional application No. 60/797,680, filed on May 5, 2006.

(51) Int. Cl. A61B 5/1473

(52) U.S. Cl. 600/347; 600/345

(57) ABSTRACT

The invention provides a voltaic cell for implantation into the body of a subject that oxidizes oxidizable biological material to generate current to do work. The voltaic cell comprises a biologically inert shell having an inner compartment containing a cathodic environment and an anodic environment on the outer surface of the shell that is in contact with bodily fluid. A connector connects the anodic environment to the cathodic environment and has a component that provides resistance between the anodic and cathodic environments. The shell contains at least one salt bridge disposed within the shell that permits passage of small ions between the inner compartment and the bodily fluid, thereby completing the circuit. The invention also provides devices such as a glucometer which continuously detects glucose and transmits a signal to an external device which provides an output, such as blood glucose level. Methods of using the same are also provided.

### CROSS-REFERENCE TO RELATED APPLICATIONS

This Application claims benefit of U.S. Provisional Application No. 60/797,680, filed May 5, 2006, the disclosure of which is hereby incorporated by reference in its entirety.

### FIELD OF THE INVENTION

The invention related to an implantable battery device which operates on an oxidizable biological material. More specifically, the invention relates to a voltaic cell that generates current through the oxidation of glucose. The device is generally applicable to implantable medical devices.

### BACKGROUND OF THE INVENTION

According to the Centers for Disease Control (CDC), there are over 20 million diabetics in the United States alone, and this number is growing at an estimated rate of 11% per year. According to the CDC, billions of dollars are spent annually on treatment of complications resulting from poor diabetic management. The medical literature has shown that strict management of blood sugar within a specific (normoglycemic) range is critical to preventing short-term to mid-term diabetic complications, (e.g., diabetic ketoacidosis) as well as long-term complications (e.g., blindness from diabetic retinopathy, amputation, cardiovascular disease and kidney failure). Standard good management, however, currently requires patients to subject themselves to several painful finger lances a day. Many diabetic patients describe “feeling like a pin cushion,” and find this daily monitoring regimen, at best, unpalatable, and in some cases, intolerable.

In many cases, diabetic patients are embarrassed to have to publicly monitor their blood sugar in order to manage their diabetes and the sight of finger-sticking and blood expression makes others uncomfortable, often fearing exposure to communicable disease. Some cultures associate a stigma with diabetes to the point that patients do not want family, friends or neighbors knowing their status as diabetics, for fear of being stigmatized. This often leads to inadequate monitoring of blood sugar and risk of complications associated with poor diabetic management. This greatly contributes to increased morbidity and mortality in the diabetic population. The fear of stigmatization can be particularly acute for teenagers, often resulting in a sudden deterioration of diabetes care with the onset of adolescence and many teenage diabetic deaths each year.

Diabetics, therefore, have long-awaited a simple, convenient, non-invasive, and discrete means to monitor blood sugar.

Several methods are currently available to measure blood glucose, with the most prevalent being finger lancing. Finger lancing involves stabbing of the finger with a small lancet and expressing a drop of blood from the wound which is then blotted onto a test strip (which is then fed into an external glucometer) or the drop of blood is fed directly into the external glucometer. This regimen is painful, inconvenient, and draws attention to the patient's diabetic status.

Other diabetic management devices include insulin pumps. Insulin pumps are essentially glucometers worn by the patient that have a plastic tube that runs directly through an open hole in the patient's abdomen and allows continuous sampling of the glucose levels in the patient's body fluids. The insulin pump continuously monitors the glucose level and injects appropriate insulin doses as determined by a computer program within the insulin pump. This theoretically allows for smooth insulin dosing and good diabetic management. However, there is a possibility that the device could malfunction and expose the patient to serious complications or death from hypoglycaemia (low blood sugar). There is also a risk of infection. These devices are expensive, may not be covered by a patient's insurance, and must be regularly maintained, serviced and calibrated.

There remains a need in the art for an inexpensive, implantable glucose meter that reliably provides real time monitoring of a patient's blood glucose concentration.

### SUMMARY OF THE INVENTION

The invention provides an implantable glucometer for monitoring blood glucose level without the need for daily finger-lancing. The solution to the problem provides for the more general invention of a battery powered by biological material within bodily fluids.

Thus, the invention provides a voltaic cell that is powered by an oxidizable biological material. As such the invention provides a voltaic cell for use in the body of a subject comprising a biologically inert shell defining an inner compartment, where the inner compartment contains an ionic solution that mediates a reduction reaction. The inner compartment contains at least one cathode (cathodic environment) within the ionic solution and the shell has at least one anode (anodic environment) attached to its outer surface that is in contact with bodily fluid of the subject when implanted. The anode accepts electrons from the oxidation reaction when the biological material is oxidized. The voltaic cell has a connector, such as a wire, that connects the anode(s) to the cathodes(s) and has a component that provides resistance between the anode and cathode. The shell also contains at least one salt bridge disposed within the shell that permits the passage of ions between the inner compartment and the bodily fluid, thereby completing the circuit.

The oxidizable biological material may be any biological material found in the subject that can be oxidixed. For example, but not by way of limitation, the material may be glucose, carbohydrates, cholesterols, fatty acids, amino acids, polypeptides, lipids and polynucleotides, nucleotides, nucleotide derivatives, etc. Preferably, the material is glucose.

In some embodiments of the invention, the voltaic cell is adapted to be a sensor for foreign or infectious particles such as viruses, bacteria, parasites, and prions, or drugs such as Coumadin®, synthetic prostacyclins, etc.

In some embodiments the voltaic cell has a plurality of either anodes, cathodes or both. In some embodiments, the cathode is made of a non-magnetic metal (such as, but not limited to silver, aluminum, lead, magnesium, platinum, gold, tin oxide, titanium dioxide, tungsten, metal alloys, non-metallic alloys, semiconductors, and semi-metals). In some embodiments, the anodes are complexed with ammonia molecules to change the potential of the oxidation reaction. In some embodiments the anodes are complexed with catalysts to change the activation energy of the oxidation reaction.

The shell of the voltaic cell may be formed from a biologically inert, impermeable substance containing a plurality of salt bridges, or the shell may be made of a semi-permeable material that allows the passage of ions between the ionic solution of the inner compartment and the surrounding bodily fluid, thereby essentially functioning as a shell with a multitude of salt bridges.

Although the connector in the voltaic cell can be a wire, any connection that allows the conduction of current is a suitable connector in the invention.

In some embodiments, the anodes of the voltaic cell have an inert barrier covering them that permits only the oxidizable biological material to access the anodes to prevent biofouling of the anodes. In some embodiments, this inert layer is on the. anodes alone. In other embodiments, a coating may be applied to the outer surfaces of the shell forming an inert barrier on the anodes, allowing only small ions and the oxidizable material to access the anodes and salt bridges.

The component that provides resistance between the anode and the cathode may be a standard resistor, that provides work, or may be a device such as a microradio transmitter that converts the electrical current into radio waves, for example.

In some embodiments, the anodic environment is compartmentalized in the shell and the cathodic environment is disposed on the outer portion of the shell.

The invention also provides an implantable glucometer that allows continuous monitoring of blood glucose in a subject. The glucometer is self-sustaining as the oxidation/reduction reaction is self-perpetuating in the body. The glucometer of the invention comprises a voltaic cell of the invention adapted to use glucose as the oxidizable biological material and an extracorporeal detection device. The detection device may be placed in proximity of the implanted glucometer and detect a signal generated by the glucometer. The detected signal is automatically correlated to blood glucose levels by the device and the blood glucose level is provided as an output by the device so one may determine the blood glucose level of the subject. The monitoring may be intermittent or continuous, and may be performed in close proximity to the implanted glucometer or remotely. Further, the output from the device may be displayed by the device itself or further transmitted to one or more other devices (such as, but not limited to computer terminals, alarm devices, and telemetric devices such as cell phones, handheld wireless devices and the like).

In some embodiments, the detection device is an amperemeter that detects electric current. In some embodiments in which the voltaic cell contains a radio transmitter, the detection device is a radio receiver that detects radio waves produced by the glucometer.

The invention also provides a method of monitoring the level of a biological material in the body of a subject by implanting a voltaic cell of the invention into the subject and detecting the signal produced by the voltaic device, correlating the signal to the amount of biological material in the bodily fluid of the subject and providing an output of a value for the amount of biological material in the bodily fluid of the subject.

In some embodiments, the invention provides a method for the monitoring of blood glucose in a subject using the glucometer of the invention. The blood glucose level may be monitored intermittently or continuously. The blood glucose level may be monitored automatically.

The invention also provides a method for powering an implantable medical device in which one or more voltaic cells of the invention are operably connected to an implanted device to provide battery power to the device. The devices may be powered by a plurality of voltaic cells arranged in series or in parallel.

### BRIEF DESCRIPTION OF THE DRAWINGS

FIG. 1 shows a basic voltaic cell of the invention with a single anode and a single cathode. A simple resistor is disposed between the anode and cathode on the connector.

FIG. 2 shows a basic voltaic cell of the invention with a single anode and a single cathode. A radio transmitter is disposed between the anode and cathode on the connector.

FIG. 3 shows an embodiment of the voltaic cell powering a device in which a wire disposed between the anodes and cathodes runs to a device to provide power to the device.

FIG. 4 shows an embodiment of the invention in which the voltaic cell is shaped as a cylinder with a central passage. The voltaic cell is shaped to be adapted to be placed in a vessel. The cell contains a plurality of cathodes and anodes. The enlarged portion shows the connection between anode, cathode and connector.

FIG. 5 shows an arrangement of a lattice mesh formed from a plurality of individual voltaic cells (Panel A); a cone-shaped implant formed from the lattice mesh (Panel B); and a U-shaped tube formed from the lattice mesh (Panel C).

FIG. 6 shows anodes covered in a biologically inert layer that prevents macromolecules from bio-fouling the anodes but permits small analytes of interest to access the anodes.

FIG. 7 shows a metabolic voltaic cell in which the anodic environment comprises enzymes and cofactors including oxidases, reductases, dehydrogenases, synthases, enzymes of the citric acid cycle, and the like, as well as Complex I, Complex II and Coenzyme Q (ubiquinone). The cathodic environment comprises cytochromes c, c, and a+a3, heme-containing porphyrin rings containing iron atoms and Complex III and Complex IV. Connectors connect the anodic and cathodic environments and the connectors contain resistors. Salt bridges are disposed between the anodic and cathodic environments.

FIG. 8 shows a metabolic voltaic cell in which a metabolic environment is disposed above an anodic environment. The metabolic environment comprises enzymes and cofactors including oxidases, reductases, dehydrogenases, synthases, enzymes of the citric acid cycle, and the like, as well as Complex I, Complex II and Coenzyme Q (ubiquinone). The cathodic environment comprises cytochromes b, c, and a+a3, heme-containing porphyrin rings containing iron atoms and Complex III and Complex IV. Connectors connect the anodic and cathodic environments and the connectors contain resistors. Salt bridges are disposed between the anodic and cathodic environments. The anodic environment is separated from the metabolic environment by gated channels operably linked to a receptor for an analyte which extends into the bodily fluid. When an analyte binds to the receptor, the channel opens and electrons from the metabolic environment flow into the anodic environment.

FIG. 9 shows a metabolic voltaic cell in which a metabolic environment is the anodic environment. The metabolic/anodic environment comprises enzymes and cofactors including oxidases, reductases, dehydrogenases, synthases, enzymes of the citric acid cycle, and the like, as well as Complex I, Complex II and Coenzyme Q (ubiquinone). The cathodic environment comprises cytochromes b, c, and a+a3, heme-containing porphyrin rings containing iron atoms, and Complex IlI and Complex IV. Connectors connect the anodic and cathodic environments and the connectors contain resistors. Salt bridges are disposed between the anodic and cathodic environments. The anodic environment is contained within an impermeable layer which forms a barrier from the bodily fluid. The impermeable layer contains gated channels operably linked to a receptor for an analyte which extends into the bodily fluid. When an analyte binds to the receptor, the channel opens and oxidizable biological material flows into the metabolic/anodic environment and are oxidized.

### DETAILED DESCRIPTION OF THE INVENTION

Any references, patents, patent applications, and/or scientific literature referred to herein evidence the knowledge of those with ordinary skill in the art, and are hereby incorporated by reference in their entirety. In the event of a conflict between any such reference cited herein and the specific teachings of this specification, the specification shall control.

Various definitions are specifically provided in the specification. In general, words shall have the meaning as understood by those of skill in the art. Specifically defined words shall have the meaning given in the definition provided herein. In the event of a conflict between the plain meaning as understood in the art and that specifically provided herein, the definition specifically taught in this specification shall supersede the art-recognized definition. Headings are merely used for convenience and shall not be construed to limit the disclosure in any manner.

Standard reference works setting forth the general principles of biochemistry and electrochemistry known to those of skill in the art include Champe, P. & Harvey, R. LIPPINCOTT's ILLUSTRATED REVIEWS, 2ND ED. Lippincott, Williams and Wilkins, 1994 and Brown, et al. CHEMISTRY: THE CENTRAL SCIENCE, 10TH EDITION. Pearson Education, 2006, which are incorporated herein by reference.

As used herein, ‘oxidizable biological material’ shall mean an organic substance that occurs in the body of a subject in which the substance is subject to having the oxidation state of any of its constituent atoms made more positive.

When referring to the oxidation of glucose herein, this encompasses the oxidation state being made more positive of any atom within the glucose molecule to form any oxidized product of glucose.

When referring to ‘reduction’ herein, this encompasses the oxidation state of the oxidant (that which is reduced) being made less positive (more negative).

As used herein, “voltaic cell” refers to a battery of separated anodic and cathodic environments connected by a salt bridge. It is also called a Galvanic cell or electrochemical cell.

As used herein, “biocompatible” refers to a state in which the material is non-toxic and a subject's body does not mount an immune response against the material.

As used herein, “subject” refers to an animal, such as a mammal, preferably human.

As used herein, “anode” refers to the electrode where net oxidation occurs. A “anodic environment” refers to an oxidizing environment which serves as an anode.

As used herein, “cathode” refers to the electrode where net reduction occurs. An “cathodic environment” refers to a reducing environment which serves as a cathode.

As used herein, “salt bridge” refers to an ionically conducting passage between separate environments of an electrochemical cell. The salt bridge may contain a conducting material or gel.

As used herein, “semi-permeable” refers to a state in which a material allows certain molecules to flow through the material, but not others.

As used herein, “impermeable” refers to a state in which a material does not allow the flow of any molecules or large atoms through the material.

As used herein, “small ion” refers to H⁺, O₂ (technically, O₂ is not an ion but is encompassed by “small ion” as used herein),and any ion necessary to complete the circuit, which can flow through the salt bridge. Small ions do not include macromolecules (such as fibrin, large molecular complexes, enzymes, or cofactors) or larger ions (such as, for example, silver ions).

As used herein, “resistor” refers to an element of an electric circuit with a fixed value of resistance.

As used herein, “amperemeter” refers to a device that detects electrical current.

As used herein, “radio receiver” refers to a device that detects radiowaves emitted by a radio transmitter.

As used herein, “sugar” refers to monosaccharides, aldoses, ketoses, disaccharides, oligosaccharides, homopolysaccharides, heteropolysaccharides, glycosides, complex carbohydrates, etc.

As used herein, “glucose” refers to the monosaccharide, common hexose sugar. “Blood glucose” and “serum glucose” are used synonymously herein.

As used herein, “polypeptide” refers to a natural compound formed from amino acids and includes proteins and protein fragments.

As used herein, “polynucleotide” refers to polymerized nucleic acid and includes DNA and RNA.

As used herein, “nucleotide” refers to nucleotide, nucleosides, or derivatives of either.

As used herein, “cholesterol” refers to the sterol of the formula 10,13-dimethyl-17-(6-methylheptan-2-yl)-2,3,4,7,8,9,11,12,14,15,16,17-dodecahydro-1H-cyclopenta[a]phenanthren-3-ol (C₂₇H₄₆O) found in cell membranes and transported in the blood of animals. Enzymatic oxidation of cholesterol forms oxysterols.

As used herein “glucometer” refers to a device that measures blood glucose level of a subject. The glucometer of the present invention has an implantable portion that “detects” glucose by generating an electric current from the oxidation of glucose. An external device detects the electric current, and through manipulation of the data to convert the electric signal into a correlated value for blood glucose level provides an output that indicates the blood glucose level of the subject in real time. The glucometer may have additional functions such as alarms to indicate hypoglycemia or hyperglycemia, for example as more fully described herein.

As used herein, “glycotoxicity” refers to tissue toxicity from long-standing hyperglycemia (high blood glucose) secondary to a constellation of pathological metabolic processes, including the conversion of excess glucose to sorbitol. Over time, this and other cytotoxic metabolites irreversibly damage the retina (causing blindness), the vasculature (causing cardiovascular disease, such as stroke, myocardial infarction and impotence), and the nerves (causing diabetic neuropathy with pain or tingling of extremities and eventual anesthesia of these tissues allowing—in the absence of adequate blood flow—infections and tissue damage to accumulate unchecked, eventually resulting in gangrene and amputation).

As used herein “analyte” refers to a substance in biological fluid that can be analyzed. The analyte may be a substance naturally occurring in the body, or may be an infectious agent (e.g., a virus, parasite or bacterium) or a product of an infectious agent. It may also be a drug.

As used herein, “about” refers to ±10% of a given reference value.

The present invention provides an implanted voltaic cell that is powered by the oxidation of a biological material. As a voltaic cell, the oxidation and reduction reactions occur in separate environments.

In a specific embodiment of the invention, the voltaic cell is a glucometer. A glucometer of the invention is a blood-sugar monitoring device that transmits a diabetic subject's moment-to-moment serum glucose level to an external receiver. After initial implantation, the glucometer of the invention allows for non-invasive measurement without the need for finger lancing. The technology allows for painless glucose monitoring, can be on demand, intermittently, continuously, or assessed conveniently at regular intervals by the patient and medical staff, and can be adapted to calculate required insulin doses, particularly for patients who have difficulty calculating doses on their own. This will allow for exquisite control of blood sugar, therefore forestalling or preventing the sequelae of cumulative glycotoxicity of long-term diabetes. It will also help prevent short-term complications of hyperglycemia, such as diabetic ketoacidosis, a life-threatening spiral of metabolic pathological acidification of the blood. The onset of ketoacidosis can rapidly follow rises in blood glucose from even brief lapses in diabetes control (i.e., missed or skipped insulin doses).

The method of the invention provides for monitoring the concentration of an analyte in a subject, or powering a device using current derived from the oxidation of biological materials.

The following discusses the components of the voltaic cell of the invention in more detail.

The Shell of the Voltaic Cell. The implant is a voltaic cell in which half of the redox reaction is contained in a biologically non-reactive shell. The biological non-reactivity of the shell prevents the subject from mounting an immunological reaction against the shell (such as initiating an allergic reaction, anaphylaxis, fibrosis or immunological destruction of the voltaic cell), while allowing the separation of the anode environment from the cathode environment.

The shell is composed of a biocompatible material such as a polymer (e.g., Teflon, polyethylene glycol (PEG), polyethylene oxide (PEO), PEG/PEO, and the like), plastic treated by coating or impregnation for biocompatibility. Other organic or inorganic biocompatible non-conductive material may also be used, such as treated silicon or glycosaminoglycans/cartilaginous material (e.g., cartilage/hyaluronan) non-immunogenic or rendered so, treated non-inmmunogenic proteoglycan material, carbon-based molecular scaffolds (e.g., carbon-based nanotubules, fullerene (e.g., buckminsterfullerene structures), and the like.

The shell may be generally impermeable and have at least one salt bridge within the shell. In some embodiments the impermeable shell comprises a plurality of salt bridges. In other embodiments, the shell is semi-permeable, such that the shell's porosity functions as the salt bridge by allowing ions to traverse the shell. The semi-permeable membrane is produced from materials such as, but not limited to the following (in part depending on the biocompatibility, cost, ionic permeability profile, toxicity profile (ideally, non-toxic), and immunogenicity profile): a semi-permeable polymer, agars, a porous inorganic plate (e.g., glass, silicon, etc.), an organic membrane (e.g. carbon-based), or lipid-based membrane (charged or uncharged, to modulate membrane permeability). Depending on the material used for the shell, the semi-permeable membrane could be made porous, focally, or diffusely, to allow for small ion permeability (e.g., H⁺, O₂, and any other ion necessary to complete the circuit, but does not allow the flow of macromolecules (such as fibrin, large molecular complexes, enzymes, or cofactors) or larger ions (such as, for example, silver ions).

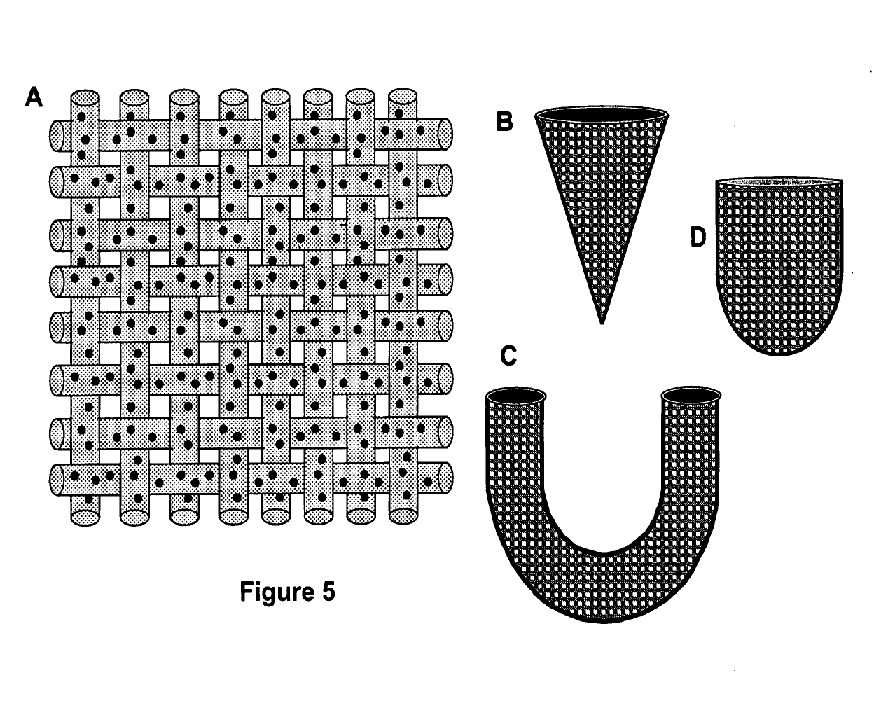

The shape of the shell (and the voltaic cell in general) is not limited, however, certain shapes may have special utilities in the body of a subject. Thus, the voltaic cell may be tubular, cylindrical, conical, toroidal, U-shaped, V-shaped, rhomboid, spherical, ovoid, or oblong or tesseract-shaped and may be formed into a cluster of cells to form a mesh, a U-shaped mesh, a conical mesh, a toroidal mesh, a tubular mesh, and the like (see FIG. 5 for illustrative embodiments).

The Cathode. The “cathode” is generally a cathodic environment. There is at least one cathode contained within the shell of the voltaic cell in contact with the ionic solution that performs the reduction half-reaction. The cathode is preferably non-magnetic and formed from a metal such as, but not limited to silver, aluminum, lead, magnesium, platinum, gold, tin oxide, titanium dioxide, tungsten, non-magnetic alloys such as stainless steel, brass, semi-metals, semi-conductors, and the like. The cathode may also be formed from a non metal provided it is able to function in the reduction reaction. In some embodiments a voltaic cell comprises a single cathode. In other embodiments, the voltaic cell comprises a plurality of cathodes or a cathodic environment that itself functions as a cathode.

The Ionic Solutions. The ionic solution of the voltaic cell may contain silverdiammine complex [Ag(NH₃)₂⁺], silver, copper, or iron salts. The use of silver in the ionic solution is more fully described below.

If the oxygen on the carbonyl group of a sugar is unattached to any other structure, the sugar is a reducing sugar, and can serve as the reducing agent of a redox pair in a reduction oxidation reaction to drive the voltaic device. Carbohydrates containing aldehydes or α-hydroxymethyl ketones can be oxidized by Cu(II) ion and are classified as reducing sugars (of which glucose and fructose are examples). They reduce Cu(II) ion to Cu(I).

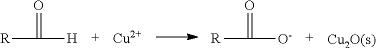

Therefore in the battery, the Ag⁺ cation of the ionic solution within the shell can be replaced by Cu²⁺, establishing a new redox pair to drive the electrogenerative reaction (this is the basis for Benedict's Test, a means of determining the presence of a reducing sugar in a body fluid or solution, as was once used in the diagnosis of diabetes mellitus). Again, a membrane with enzymatic catalysts at the anode sensor could be applied to facilitate the reaction by lowering the activation energy, but even without an enzymatic substrate, the spontaneous thermodynamics of the reaction prevail. Reducing sugars in the body fluid will be oxidized at the anode in contact with the body fluid, and electrons will flow from the sugar at the anode through a connector to reduce the copper cation at the cathode within the shell. As with other embodiments, a pore (salt bridge, etc.) can be employed to complete the circuit and a device or resister placed in the circuit to, do work, provide power or to send a signal. Modifications or complexes of Cu and catalysts can be used to change the thermodynamics, activation energy, or performance of this votalic cell.

The Anode. There is at least one anode at the outer surface of the shell in contact with the bodily fluid in which the oxidation reaction occurs. The anode is preferably formed from a non-magnetic metal such as, but not limited to silver, aluminum, lead, magnesium, platinum, gold, tin oxide, titanium dioxide, tungsten, non-magnetic alloys such as stainless steel, brass, semi-metal, semi-conductors, and the like. The anode may also be formed from a non metal provided it is able to fimction in the oxidation reaction. In some embodiments a voltaic cell comprises a single anode. In other embodiments, the voltaic cell comprises a plurality of anodes. The anodes of the invention may be complexed with an enzyme that catalyzes the oxidation of the biological material of interest. Oxidases are well-known in the art and may be appropriately selected for use in a particular system of choice. The coupling of enzymes to anodes is also known in the art and may be by any means known. For example, the enzyme may be applied in a permeable layer, which may optionally contain stabilizers. It may also be immobilized in this layer.

The Connector. The cathode(s) and anode(s) is/are connected to one another by a connector. The connector, may be a wire, for example, but may be any connection that permits the flow of current from the anode to the cathode. The connector is preferably formed from a non-magnetic conductive metal. The connector also comprises a component that provides resistance along the connection. The component may be, for example, a simple resistor. The component may be a radio transmitter, such as a micro-radio that converts electrical current into radiowaves. The component may be another device to be powered by the voltaic cell.

The Salt Bridge. The salt bridge is any opening in the shell that permits the passage of small ions. The salt bridge may be a small hole in the shell to selectively allow the passage of the small ions. The shell of the voltaic cell comprises at least one salt bridge. In some embodiments, the shell contains a plurality of salt bridges. In other embodiments the shell is semi-permeable, effectively being a shell containing a multitude of salt bridges.

Optional Elements. Optionally, the voltaic cell may further comprise an inert, biologically compatible boundary layer that is disposed over the anode(s) on the outer surface of the shell. The inert boundary layer prevents large molecules from accessing the anodes and only permits the passage of small ions to reach the anode(s). This layer forms a boundary to prevent bio-fouling of the voltaic cell. In some embodiments, the boundary layer only covers the anodes. In other embodiments, the boundary layer is applied as a layer over the surface of the shell. In such a case, the boundary layer is applied such that there is no blocking of the salt bridges of the cell. In general, any boundary layer that prevents bio-fouling may be used. The boundary layer may operate by steric hindrance, size exclusion, surface tension, mass transfer, or any other means known in the art. The boundary layer may be composed of porous polymers that exclude large macromolecules but permit the passage of glucose and other analytes, for example. Materials such as, but not limited to polypropylene, polysulphone, polytetrafluoroethylene (PTFE) and poly(ethylene terephthalate) (PET), or a lipid layer, could be employed.

The voltaic cell may also be coated with an anti-coagulant to prevent clotting of blood around the cell. Anticoagulants are well-known in the art and coating devices and implanted objects with anticoagulants is also known in the art. The anti-coagulant may be applied by bathing, spraying, washing, dipping, immobilizing chemically or otherwise into or incorporating into surface or any other part of device. The anticoagulant may also be an impregnated membrane applied to the device or any part of device. Various anticoagulants are known in the art. Heparin is an example that is suitable for use in the invention.

The voltaic cell may also be coated with an inhibitor of the deposition of collagen or other matrix proteins to prevent encapsulation of the voltaic cell. Polyethylene glycol (PEG), or phenol structures, are examples of a suitable inhibitor that may be used to inhibit the adherence of proteins from the surface of the voltaic cell.

The voltaic cell may also contain additional electronic or electrophysical components in the circuitry such as, for example, capacitors, semiconductors, transistors, rectifiers, transmitters, receivers, resistors, reference electrodes and the like, depending on the applications of the voltaic cell(s) to be implanted. One of skill in the art is well-versed in circuitry to achieve a desired effect to optimize the performance of the voltaic cell for a given application.

Optionally, a catalyst (enzymatic or otherwise) may be provided at an electrode or at any site of redox reaction to change the activation energy and/or modulate the performance of the voltaic cell. For example, oxidizing enzymes may be complexed on or near the anode of the cell. Examples of enzymes that oxidize biological materials include, but are not limited to:

| Enzyme | Analyte Oxidized |
|---|---|
| glucose oxidase | glucose |
| hexose oxidase | glucose |
| lactate oxidase | lactate |
| 1-amino acid oxidase | 1-methionine |
| 1-amino acid oxidase | 1-phenylalanine |
| D-amino acid oxidase | d-aspartate |
| D-amino acid oxidase | d-glutamate |
| uricase | urate |
| cholesterol oxidase | cholesterol |

Thus, non-limiting examples of enzymes that may be complexed to the anodic portion of the voltaic cell include glucose oxidase, cholesterol oxidase, hexose oxidase, 1-amino acid oxidase, D-amino acid oxidase, uricase, lactate oxidase, choline oxidase, D-amino acid oxidase, alcohol oxidase, uricase, xanthine oxidase, bilirubin oxidase, glutamate oxidase, putrescine oxidase and polyamine oxidase. Further, an optional redox mediator/limiter/amplifier may be provided, as needed or desired.

External Devices. The voltaic cell is implanted in the body of a subject, preferably under the skin to allow easy access for external devices to detect signals generated by the voltaic cell. In general, the external devices detect a signal from the voltaic cell and provide an output. In some embodiments, the signal that is detected is current. In other embodiments, the signal that is detected is a radio transmission. In other embodiments, the signal is magnetic flux (which allows the external device to query the implanted device through inductance). In other embodiments the signal is telemetrically received from the implanted voltaic cell. The external devices may include circuitry to calibrate, filter, clean, smooth, amplify or store the data received from the device. The external devices may also analyze the data received and calculate the concentration of the analyte detected by the voltaic cell. For example, the external device may calculate the concentration of blood glucose detected by the voltaic cell based on programmed calculations and/or algorithms performed by the external device. The external device may store the data or further transmit data to other devices (such as a computer, monitor, alarm, cell phone, wireless hand-held devices (e.g., Blackberry™)). The functions of the external device are not limited and may be any that are appropriate and useful for the application of the voltaic cell.

In some embodiments, the external device is an amperemeter. In other embodiments the external device is a radio receiver. The external device may monitor the voltaic cell signal on demand, intermittently or continuously. The external device may be placed in close proximity to the voltaic cell (such as by waving a hand-held external device over the area of the subject where the implanted voltaic cell resides). The external device may also be worn by the subject and may take the form of a wristwatch-like device, a collar (particularly for animal subjects), belt or other form conveniently attached to the subject. The external device may also be at a more remote location such as a wall-mounted, tabletop or other station which is close enough to the subject to detect the signal from the voltaic cell. Such devices may be conveniently placed near subjects in a hospital, clinic, nursing home, classroom, home, kennel, and the like. Such devices allow the monitoring of subjects, particularly when the subject cannot reliably monitor themselves (particularly children, the elderly, handicapped, incapacitated, mentally-challenged, and animal subjects).

The external devices may provide a reading of the concentration of the analyte to be measured (e.g., blood glucose) and may include an alarm, instructions for taking a medication based on the reading (e.g., insulin administration), and the like. As such, the voltaic cell may be coupled with a device that administers drug based on the readings obtained by the device. The drug administration device may be coupled to the voltaic cell or separately connected to the subject and respond to a signal generated from the external device.

The voltaic cells of the invention may be used as batteries to power other implanted devices. The voltaic cells may be operably connected to the implanted devices and powered using a ready source of biological material (e:g., glucose). Voltaic cells may be arranged in parallel to increase capacitance and maintain voltage or in series to increase the voltage while maintaining capacitance, depending on the desired application.

Analytes to be Measured. The principles of the voltaic cell of the invention have broad applicability and may be used as a device to convert the oxidation of a biological material into a detectable signal, and thus, the voltaic cell of the invention may be employed as a detection device for a wide variety of analytes. The voltaic cells of the invention may detect at least one analyte and may be configured to detect a plurality of analytes. The analytes to be detected include any biological material that can be oxidized at the anode of the voltaic cell. The biological materials include, but are not limited to glucose, sugars, fatty acids, cholesterol, lipids, polynucleotides, amino acids (such as phenylalanine in PKU patients); polypeptides (including for example, hormones such as estrogen, progesterone, testosterone, growth hormone, thyroid hormone and the like (which can be monitored, for example, during hormone therapy, pregnancy, or to detect ovulation); cancer-specific protein markers of cancer cells which can be monitored to detect metastasis or relapse; early markers of disease such as prostate-specific antigen (PSA); and the like) The analytes may also be a drug, such as Coumadin® or synthetic prostacyclins, etc.

Further, through adapting the voltaic cell to provide a gated channel that opens to expose the anode(s) to an analyte to generate current only when binding of a specific analyte to a receptor operably attached to the channel, the voltaic cell of the invention is suitable for detection of foreign or infectious particles, including, for example, viruses, bacteria, parasites and prions. The voltaic cell may be employed, for example to monitor viral load in HIV and other viruses, monitor bacterial/parasitic infection and antibiotic treatment, and the like.

In one embodiment, for example, a transmembrane spanning protein comprises a channel operably linked to a receptor for viral protein of interest. The membrane with embedded protein coats the anode and prevents the accession of a detectable analyte (such as glucose). When the circulating viral protein reversibly binds to the receptor, a conformational change occurs in the channel and allows glucose to access the anode where it is then oxidized. Current is generated in the voltaic cell, and the external device, which is precalibrated to convert the signal (e.g., current or radiowaves if a radio transmitter is used) to an indication of viral protein concentration, provides a useful output indicating viral load in the subject.

The voltaic cell of the invention may also be adapted as a sensor for drugs in the bloodstream of a subject. The sensor may detect at least one drug or metabolite thereof an transmit the data to the external device. This embodiment is useful in monitoring a subject for safe levels of therapeutic drugs such as Coumadin®, Epoprostenol, pain management drugs and anti-cancer therapies, as well as to monitor the abuse of drugs. In these embodiments, the voltaic cells containing receptors are used in which the receptor detects the presence of the drug or metabolite thereof.

General Principles of the Voltaic Cell. A voltaic cell generates current through a redox (reduction-oxidation) reaction, the voltage generation and resultant electric current of which are described by the Nernst equation:

E=E⁰−RT/nF ln Q_{c}

where E is the potential (i.e., the voltage) generated from the reaction, E⁰ is the standard potential between the two electrodes, R is the universal gas constant, T is the absolute temperature, n is the charge number of the electrode reaction (number of moles of electrons in the reaction), and F is the Faraday constant. Q_{c} represents the ratio of the concentration of the chemical species appearing on the reduced side of the electrode reaction to the concentration of the chemical species appearing on the oxidized side of the electrode reaction (the In notation implies the potential generated is related to the natural logarithm of this ratio).

The redox reaction providing the mechanism for electric current generation from serum glucose in the proposed voltaic cell is based on the following oxidation potential of glucose and reduction potential of Ag⁺:

$$\frac{\begin{matrix}2 {Ag ( {NH}_{3} )}_{2}^{+} + {e}^{-} \to Ag + 2 ( {NH}_{3 } ) & {E}_{red}^{0} = 0.373 V \\ + {C}_{6} {H}_{12} {O}_{6} + {H}_{2} O \to {C}_{6} {H}_{12} {O}_{7} + 2 {H}^{+} + 2 {e}^{-} & {E}_{ox}^{0} = 0.600 V\end{matrix}}{\begin{matrix}2 {Ag ( {NH}_{3} )}_{2}^{+} + {C}_{6} {H}_{12} {O}_{6} + \\ {H}_{2} O \to 2 Ag + {C}_{6} {H}_{12} {O}_{7} + 2 {NH}_{4}^{+}\end{matrix} {E}^{0} = 0.973 V}$$

This reduction-oxidation pairing is known as the Tollens reaction, and is an ancient means of creating a mirrored surface from ionic silver in solution.

The half potentials of voltaic reactions can change when either of the species of the redox pair is modified. For example, the half cell potential of the simple reduction of silver cations (Ag⁺) is the following:

Ag⁺+e⁻→Ag E^{o}_{red}=0.800 V

and the half cell potential of the simple reduction of the glucose molecule is the following:

C₆H₁₂O₆+H₂O→C₆H₁₂O₇+2 H⁺+2 e⁻ E^{o}ₐₓ=−0.050 V

Even though the simple oxidation of the glucose molecule appears to be thermodynamically unfavorable (non-spontaneous because of the negative sign), the overall cell potential of the full reaction, 0.750 V, is thermodynamically favorable and spontaneous, given the overall positivity (positive sign) of the full reaction.

When the silver ion is complexed with NH₃, for example, as is the case with the glucometer of the invention the half cell potential of the reduction of the silver diammonia cationic complex [Ag(NH₃)₂⁺] differs dramatically:

Ag(NH₃)₂⁺+e⁻→Ag+2NH₃ E^{o}_{red}=0.373 V

The reaction of the modified species, though still spontaneous and thermodynamically favorable, has a significantly smaller magnitude than that of its pure silver cation cousin.

If the reaction were occurring all in one flask, the presence of NH₃ molecules would increase the pH of the overall reaction solution, thereby also affecting the half cell potential of the oxidation of glucose. This follows logically from Le Châtelier's Principle. Examining the oxidation reaction of glucose, one can see that the right side of the equation contains H⁺ (acidic) ions; thus, allowing the reaction to proceed in a high pH milieu (e.g. increased basicity from the presence of NH3 molecules) means that H⁺ ions will be consumed, and the reaction will be driven to the right. This should mean increased thermodynamic favorability of the oxidation of glucose in the presence of ammonia, and indeed, this is observed:

C₆H₁₂O₆+H₂O→C₆H₁₂O₇+2 H⁺+2 e⁻ E^{o}ₒₓ=0.600 V

As can be seen, the oxidation reaction of glucose is now positive (spontaneous, thermodynamically favorable) in a basic (high pH) milieu. The overall potential of the full redox reaction of the silver complex and glucose in the presence of ammonia is the following:

2Ag(NH₃)₂⁺+C₆H₁₂O₆+H₂O→2Ag+C₆H₁₂O₇+2NH₄⁺ E^{o}=0.973 V

Not only is this still thermodynamically favorable, the cell potential of this reaction is significantly increased compared to that of the redox reaction of the simple silver cation and glucose redox pair.

These are the general principles of the redox reactions which govern the electric potential of the cell. One of skill in the art is well-versed in the ionic solutions and anode/cathode compositions suitable to produce voltaic cells in accordance with the invention. The standard state cell potentials of half reactions are well known as are ionic solutions and anode/cathodes that would be suitable for use in the invention. The standard-state cell potentials for some common half-reactions are listed in Table 1.

TABLE 1

| Half-Reaction | E°_{red} |
|---|---|
| K⁺ + e⁻ [symbol] K | −2.924 |
| Ba²⁺ + 2 e⁻ [symbol] Ba | −2.90 |
| Ca²⁺ + 2 e⁻ [symbol] Ca | −2.76 |
| Na⁺ + e⁻ [symbol] Na | −2.7109 |
| Mg²⁺ + 2 e⁻ [symbol] Mg | −2.375 |
| H₂ + 2e⁻ [symbol] 2 H⁻ | −2.23 |
| Al³⁺ + 3 e⁻ [symbol] Al | −1.706 |
| Mn²⁺ + 2 e⁻ [symbol] Mn | −1.04 |
| Zn²⁺ + 2 e⁻ [symbol] Zn | −0.7628 |
| Cr³⁺ + 3 e⁻ [symbol] Cr | −0.74 |
| S + 2 e⁻ [symbol] S²⁻ | −0.508 |
| 2 CO₂ + 2 H⁺ + 2 e⁻ [symbol] H₂C₂O₄ | 0.49 |
| Cr³⁺ + e⁻ [symbol] Cr²⁺ | −0.41 |
| Fe²⁺ + 2 e⁻ [symbol] Fe | −0.409 |
| Co²⁺ + 2 e⁻ [symbol] Co | −0.28 |
| Ni²⁺ + 2 e⁻ [symbol] Ni | −0.23 |
| Sn²⁺ + 2 e⁻ [symbol] Sn | −0.1364 |
| Pb²⁺ + 2 e⁻ [symbol] Pb | −0.1263 |
| Fe³⁺ + 3 e⁻ [symbol] Fe | −0.036 |
| 2 H⁺ + 2 e⁻ [symbol] H₂ | 0.0000 . . . |
| S₄O₆²⁻ + 2 e⁻ [symbol] 2 S₂O₃²⁻ | 0.0895 |
| Sn⁴⁺ + 2 e⁻ [symbol] Sn²⁺ | 0.15 |
| Cu²⁺ + e⁻ [symbol] Cu | 0.158 |
| Cu²⁺ + 2 e⁻ [symbol] Cu | 0.3402 |
| O₂ + 2 H₂O + 4 e⁻ [symbol] 4 OH⁻ | 0.401 |
| Cu⁺ + e⁻ [symbol] Cu | 0.522 |
| I₃⁻ + 2 e⁻ [symbol] 3 I⁻ | 0.5338 |
| MnO₄⁻ + 2 H₂O + 3 e⁻ [symbol] MnO₂ + 4 OH⁻ | 0.588 |
| O₂ + 2 H⁺ + 2 e⁻ [symbol] H₂O₂ | 0.682 |
| Fe³⁺ + e⁻ [symbol] Fe²⁺ | 0.770 |
| Hg2²⁺ + 2 e⁻ [symbol] Hg | 0.7961 |
| Ag⁺ + e⁻ [symbol] Ag | 0.7996 |
| Hg²⁺ + 2 e⁻ [symbol] Hg | 0.851 |
| H₂O₂ + 2 e⁻ [symbol] 2 OH⁻ | 0.88 |
| HNO₃ + 3 H⁺ + 3 e⁻ [symbol] NO + 2 H₂O | 0.96 |
| Br₂(aq) + 2 e⁻ [symbol] 2 Br⁻ | 1.087 |
| 2 IO₃⁻ + 12 H⁺ + 10 e⁻ [symbol] I₂ + 6 H₂O | 1.19 |
| CrO₄²⁻ + 8 H⁺ + 3 e⁻ [symbol] Cr³⁺ + 4 H₂O | 1.195 |
| Pt²⁺ + 2 e⁻ [symbol] Pt | 1.2 |
| MnO₂ + 4 H⁺ + 2 e⁻ [symbol] Mn²⁺ + 2 H₂O | 1.208 |
| O₂ + 4 H⁺ + 4 e⁻ [symbol] 2 H₂O | 1.229 |
| Cr₂O₇²⁻ + 14 H⁺ + 6 e⁻ [symbol] 2 Cr³⁺ + 7 H₂O | 1.33 |
| Cl₂(g) + 2e⁻ [symbol] 2Cl⁻ | 1.3583 |
| PbO₂ + 4 H⁺ + 2 e⁻ [symbol] Pb²⁺ + 2 H₂O | 1.467 |
| MnO₄⁻ + 8 H⁺ + 5 e⁻ [symbol] Mn²⁺ + 4 H₂O | 1.491 |
| Au⁺ + e⁻ [symbol] Au | 1.68 |
| H₂O₂ + 2 H⁺ + 2 e⁻ [symbol] 2 H₂O | 1.776 |
| Co³⁺ + e⁻ [symbol] Co²⁺ | 1.842 |
| S₂O₈²⁻ + 2 e⁻ [symbol] 2 SO₄²⁻ | 2.05 |
| O₃(g) + 2 H⁺ + 2 e⁻ [symbol] O₂(g) + H₂O | 2.07 |
| F₂(g) + 2 H⁺ + 2 e⁻ [symbol] 2 HF(aq) | 3.03 |

With reference to the glucometer version of the voltaic cell, in some embodiments, the reaction is allowed to proceed spontaneously (without anmmonia) with an overall cell potential of 0.750 V. However, in practice, this may tend to result in the formation of colloidal silver spheres in solution, rather than the uniform and controlled plating of silver onto the cathode. In addition, it is desirable to have NH₃ in cathodic solution as Ag₂O (silver (I) oxide) can be created when elemental silver at the cathode is oxidized by molecular oxygen diffusing into the cathodic environment, over the salt bridge. Ag₂O is minimally soluble in water (0.0013 g/100 ml (20° C.) to 0.0017 gm/100 ml water) but is more soluble in aqueous solutions of alkali hydroxides, because of the formation of the ion, Ag(OH)₂⁻. By adding NH₃ to the cathodic environment, some portion of the NH₃ will react with H₂O to form ammonium hydroxide (NH₄OH), maintaining the oxidized silver in solution to reenter the cycle of reduction at the cathode, thus helping perpetuate the reaction. It is unclear whether the formation of colloidal silver would interfere over time with the function of the device, but, without wishing to be bound to any particular theory of operability, it is presently believed that silver metal plating onto the cathode is preferable.

In other embodiments, the reduction of, and plating of silver onto the cathode is employed. In these embodiments, the ionic solution within the shell contains a basic compound such as containing high ammonia. Other basic species may be used, but this will likely change the thermodynamics of the reaction, such that one of skill in the art would select the solutions to suit the particular application, and such skill is well within the ambit of the skilled artisan. The glucose oxidation half reaction occurs in the near neutral pH of the blood. This results in an overall cell potential of about 0.323 V. This is still a spontaneous reaction, although the thermodynamic favorability of this reaction is of lower magnitude.

In other embodiments of the invention, a membrane is deposited or incorporated at the anode that is impregnated with or complexed with NH₃ molecules or some other molecular species to mediate the thermodynamics of the redox reaction. In the case of NH₃, for example, complexing this species to the anode (or the membrane incorporated with the electrode) would result in a higher overall cell potential: 0.973 V.

In other embodiments, the anode(s) could have NH₃ complexed to its (their) surface(s) (or some other basic molecule or ion) to allow the oxidation of glucose to occur in a high pH environment, but allow for the formation of colloidal silver by not providing ammonia inside the shell. This results in a predicted overall cell potential of the reaction of 1.4 V.

Thus, it is possible to modulate the potential of the half reactions by modifying the pH of the milieu at the anode, the cathode, neither, or both.

Glucose is an energy-rich biological material that is well suited for the voltaic cell of the invention. However, the invention is not limited to harnessing energy through the oxidation of glucose. The body has many energy-rich materials that can be used by adapting the voltaic cell of the invention, and the power of any energy-rich biological molecule can be harnessed to provide electrical energy by separating biochemical redox pairs. If the electrons that are naturally transferred among biological redox pairs are allowed to flow through a connector, a current is generated.

The metabolism of biological materials are facilitated by well-understood enzymatic catalysts and biochemical reactions. These include, for example:

(a) fatty acids (triacylglycerols, prostaglandins, steroids, etc.) through β-oxidation, decarboxylation, hydrolysis/hydroxylation, etc.

(b) )carbohydrates (monosaccharides, aldoses, ketoses, disaccharides, oligosaccharides, homopolysaccharides, heteropolysaccharides, glycosides, complex carbohydrates, etc.) through hydrolysis, conversion to acetyl CoA, oxidation, glycolysis, the citric acid cycle, etc.

(c) amino acids (glucogenic, ketogenic; oxaloacetate-generating; α-ketoglutarate-generating; pyruvate and fumarate-generating; succinyl Co, acetyl CoA, and acetoacetyl CoA-generating, etc.), and proteins (including glycoproteins, lipoproteins, etc.) through transamination, deamination/oxidative deamination, oxidative decarboxylation, dehydrogenation, etc.; and

(d) other energy-rich biological molecules or metabolites such as glycerols (from the mobilization of stored fat, etc.), pyruvate/lactate, ATP/ADP, ketone bodies (acetoacetate, β-hydroxybutarate, acetone), through oxidation, hydrolysis, conversion, etc.

The metabolism of these molecules donates electrons to nicotinic adenine dinucleotide (NAD⁺) and flavin adenine dinucleotide (FAD) through (reductases, dehydrogenases, synthases, etc.) to form the reduced, electron (and energy) rich molecules of NADH and FADH₂, respectively.

These reduction oxidation reactions are the first step of the body's primary means of creating ATP (a major energy currency molecule used to perform work in the cells). Once NADH and FADH₂ have been created from metabolism of fatty acids, amino acids and carbohydrates (and other substrates, comprising but not restricted to those above), they donate their electrons down the electron transport chain through coenzymes to oxygen, an avid electron acceptor. This forms the basis of oxidative phosphorylation, or cellular respiration, by which the oxidation of fatty acids, amino acids and carbohydrates to CO₂ and H₂O transfers electrons to O₂, which is reduced to H₂O.

This forms the basis of a biological redox pair for use in the voltaic device. The anode would comprise immobilized, well described, reversible enzymes for the metabolism of fatty acids, amino acids and carbohydrates (reductases, dehydrogenases, synthases, enzymes of the citric acid cycle, etc.), and Complex I, Complex II and coenzyme Q of the inter-membrane space of the mitochondria, in contact with the body fluid:
Complex I: Comprises NAD⁺/NADH, FMN/FMNH₂, NADH dehydrogenase
Complex II: Comprises NAD⁺/NADH, FAD/FADH₂, fumarate/succinate, succinate dehydrogenase, acyl CoA dehydrogenase
Coenzyme Q: A quinine derivative (also known as ubiquinone, because of its ubiquity in living systems) oxidizes (takes electrons from) both FMNH₂ and FADH₂ and transfers them to Complexes IIII and IV.
   The hydrogen ions (cationic and anionic) are readily available in the blood stream and small enough to perfuse the reaction arena to allow for the transfer of electrons through the trading of hydrogen ions between reactants. The FMN/FMNH₂ pair is a reversible intermediate in the electron transport chain that oxidizes NADH to NAD⁺ (by reducing FMN to FMNH₂). The electrons from this redox reaction are then transferred from FMNH₂ to Coenzyme Q, through the oxidation of FMNH₂ to FMN and the reduction of CoQ to CoQH₂. Likewise, FADH₂ is oxidized to FADH to reduce CoQ to CoQH₂.

Complexes III and IV, and cytochrome c of the electron transport chain would make up the cathode. The components of this system of complexes are the cytochromes b, c, and a+a₃, containing heme groups made of porphyrin rings containing an iron atom that is reversibly converted from its ferric (Fe³⁺) to its ferrous state (Fe²⁺) through redox reaction, thus serving as a reversible carrier of electrons.
Complex III: Comprises Cytochrome b, which receives electrons from (is reduced by) Coenzyme Q, oxidizing CoQH₂ to CoQ.
Cytochrome c: Transfers electrons from Complex III to Complex IV
Complex IV: Comprises cytochrome a+a₃ and accepts electrons from (is reduced by) cytochrome c, oxidizing the metals groups of cytochrome c. Cytochrome a+a₃ is the only cytochrome in which the heme iron has a free ligand that can readily react with O₂. This cytochrome also contains bound copper atoms, required for the reaction. Electrons are transferred to O₂, reducing it to H₂O, which oxidizes the metal groups of cytochrome a+a3.
   As in other embodiments, the O₂ molecule (and hydrogen ions) will naturally perfuse through the pores/salt bridges of the shell and be available for reduction to H₂O at the cathode. The O₂ molecule will not be appreciably reduced at the anode outside the shell, though oxygen is richly available there, because (1) there is no cytochromic iron moiety free ligand to react with it, and (2) the electron transport chain is downhill; once the electrons have been passed via hydrogen ions to subsequent intermediaries of the chain, they cannot travel backward up the chain as this is thermodynamically unfavorable. Electrons will have flowed down to cytochrome a+a₃ providing a ready supply of raw electrons to react with oxygen at the cathode. By separating the redox pair and facilitating the irreversible flow of electrons into the initial components of the electron transport system, oxygen at the cathode becomes an electron well, and is readily available there to receive them.

As in other embodiments, the anodic environment in contact with the blood stream is separated from the cathodic environment and joined by a connector. The flow of electrons through the connector generates a current. The cathodic environment should be impermeable to components of Complexes I and II, and ideally metabolic intermediaries, though O₂ and small ions such as protons and Cl⁻, should be able to pass freely.

The Nernst thermodynamics of this reaction are favorable, which means it is spontaneous and can be harnessed to generate electric power:

| Redox Reaction | Eₒ (Standard Reduction Potential) |
|---|---|
| NADH → NAD⁺ | 0.32 |
| FADH₂ → FAD | 0.22 |
| ½ O₂ → H₂O | 0.82 |
| Total Reaction | 1.36 |

The voltaic cell of the invention may be constructed compartmentalizing the anodic environment, comprising electron donors such as NADH, FADH₂, FMNH₂, antioxidants such as glutathione or ascorbate (ascorbic acid, or vitamin C), or active reductants such as Sodium borohydride (NBH₄). The cathodic environment would now be in contact with the blood stream, whereby the voltaic is powered by reduction of oxidants in the blood stream (or body fluid), with electrons passing from the electron donors/antioxidants/reductants through the connector to the oxidants, thus generating a current. In this embodiment, it would be ideal to sequester the anodic environment from more electronegative species such as O₂ or oxygen radicals, H₂O₂, nitrogen oxide species (.NO₂/.NO), Fe³⁺, Ca³⁺, Mg³⁺, etc., in order to allow the orderly flow of electrons through the connector.

As is well known in the art, a voltaic cell can also be run backward to re-oxidize oxidants previously used in the cathodic environment. This is referred to as electrolysis, whereby an outside power source, for example, in this case magnetic inductance, is used to pump electrons “uphill.” When this reverse reaction begins, what is referred to as the “cathodic environment” and the “anodic environment” are now reversed, by convention. For clarity, we remember electrons always move from the anode toward the cathode, in both cases.

Certain embodiments of the invention will now be described with reference to the drawings.

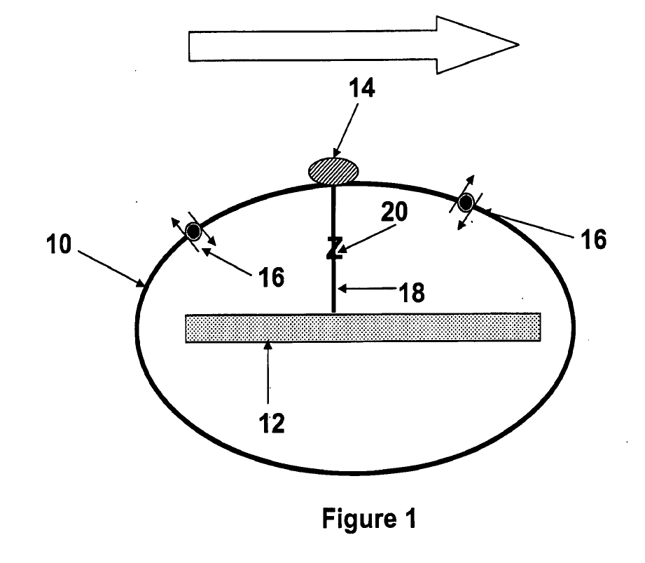

A voltaic cell, in basic format, is shown in FIG. 1. The voltaic cell has a biocompatible shell 10 defining a compartment which encloses a cathode 12 in an ionic solution. The shell 10 has at least one salt bridge 16 that allows the passage of ions between the ionic solution and the bodily fluid outside the shell 10. The voltaic cell also has at least one anode 14 positioned on the outer surface of the voltaic cell. The anode 14 is connected to the cathode 12 by a connector 18 (such as a wire). A component that provides resistance 20 is positioned between the anode and the cathode. The large arrows represent bodily fluid surrounding the voltaic cell.

In practice, the exterior surface of the implanted cell will be in contact with bodily fluid (e.g., blood) of the subject. The blood glucose is oxidized at anode 14 and electrons flow down the connector 18 to the cathode 12. Ag⁺ ions in the ionic solution within the shell are reduced and plated onto the cathode 12. Salt bridges 16 are provided that allow small ions to flow through the shell to complete the circuit. In this environment, oxygen diffuses across the salt bridge 16 and reoxidizes the silver at cathode 12, and the silver returns to the ionic solution as Ag⁺/[Ag(NH₃)₂⁺]. As the reactions occur, electrons flow from the anode 14 to the cathode 12 along the connector 18. A device providing resistance (such as a resistor) 20 is disposed along the connector 18. As current flows through the voltaic cell, an external device positioned outside of the body of the subject can detect the current.

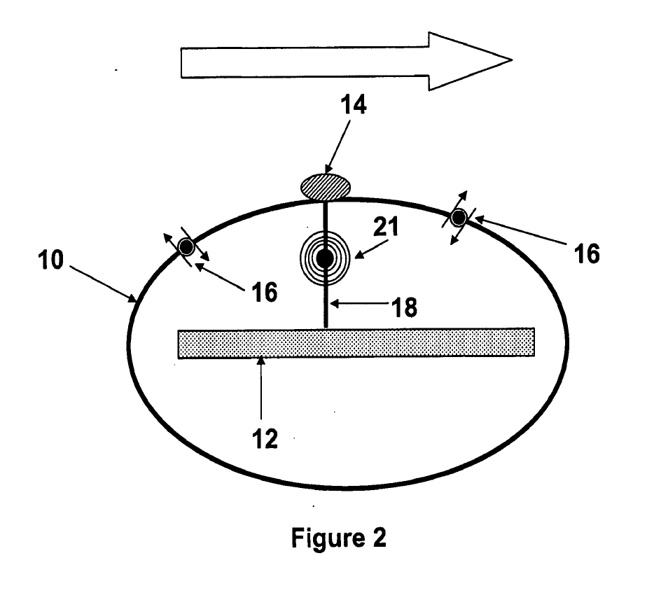

In another embodiment shown in FIG. 2, a voltaic cell, in basic format, is shown. The voltaic cell has a biocompatible shell 10 defining a compartment which encloses a cathode 12 in an ionic solution. The shell 10 has at least one salt bridge 16 that allows the passage of small ions between the ionic solution and the bodily fluid outside the shell 10. The voltaic cell also has at least one anode 14 positioned on the outer surface of the voltaic cell. The anode 14 is connected to the cathode 12 by a connector 18 (such as a wire). In this case, the component that provides resistance 21 is a micro radio transmitter positioned between the anode and the cathode. The large arrow represents passage of bodily fluid surrounding the voltaic cell. In practice, the current flowing through the voltaic cell is converted to radiowaves which are emitted from the device. These radiowaves can be detected using a radio receiver which is positioned outside of the body of the subject.

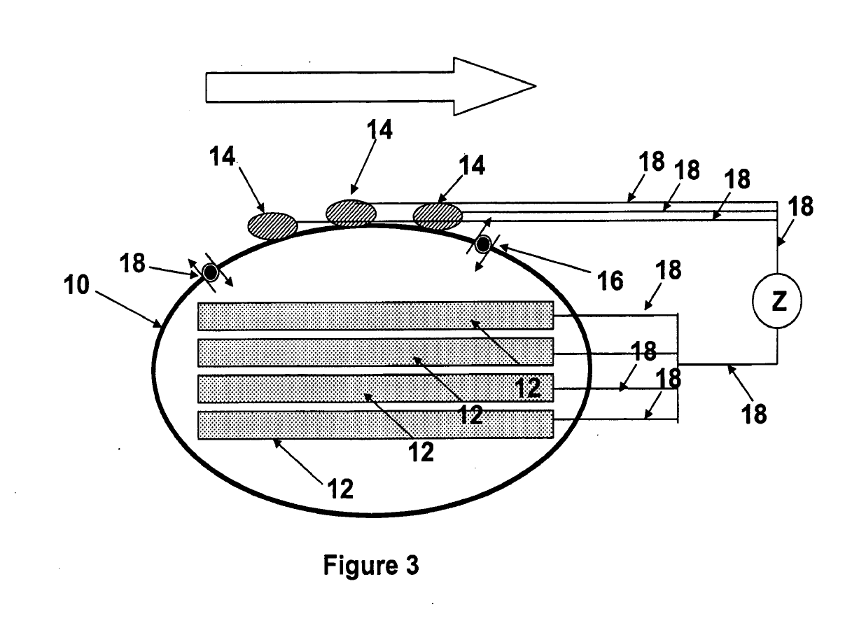

FIG. 3 shows another embodiment of the voltaic cell used as a battery containing an impermeable shell 10, a plurality of anodes 14, a plurality of cathodes 12, salt bridges 16, and connectors 18 connecting the anodes 14 and cathodes 12. The anodes and cathodes are connected by connectors 18 that have a device disposed along the connector that provides resistance 20 and is powered by the voltaic cell.

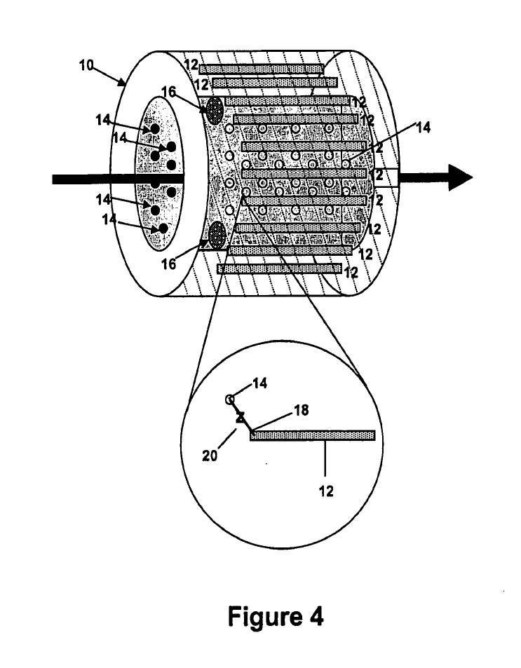

FIG. 4 shows an embodiment of the voltaic cell adapted for use in a vessel. The shell 10 has at least one salt bridge 16 that allows the passage of small ions between the ionic solution and the bodily fluid outside the shell 10. The shell 10 is shaped as a tube comprising a cylinder with a central passage for blood flow. The interior compartment of the shell contains a plurality of cathodes 12. The passage contains a plurality of anodes 14 positioned on the surface of the shell 10 positioned on the aspect in contact with the flow of blood. The inset shows that cathodes 12 are connected to anodes 14 by a connector 18 having an element providing resistance 20 on the connector 18. The voltaic cell has salt bridges 16 disposed on the shell 10 to allow flow of ions between the blood stream and the inner compartment containing the ionic solution.

FIG. 5 shows an embodiment in which a plurality of voltaic cells is interwoven to create a lattice mesh (Panel A). Each voltaic cell of the mesh comprises a plurality of anodes (represented by black circles) which are connected to internal cathodes by connectors. The mesh may be implanted directly into the body of the subject or formed into a particular shape, adapted for implantation at a desired site within the body. FIG. 5 (Panel B) shows a cone-shaped device formed from the mesh. FIG. 5 (Panel C) shows a U-shaped tube device formed from the mesh. FIG. 5 (Panel D) shows a cup-shaped tube device formed from the mesh.

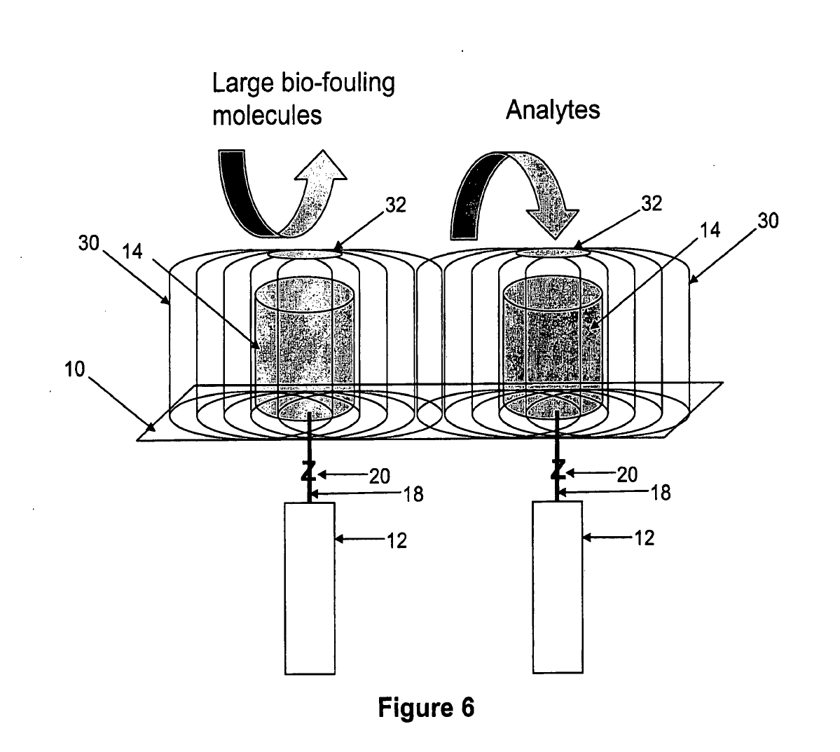

FIG. 6 shows view of anodes 14 on the surface of a shell 10 wherein the anodes 14 are covered with a biologically inert boundary material 30 that forms a barrier to large bio-fouling molecules, but permits small molecules and analytes to enter pores 32 and contact the anodes 14. The anodes 14 are connected to cathodes 12 with connectors 18 which have resistors 20.

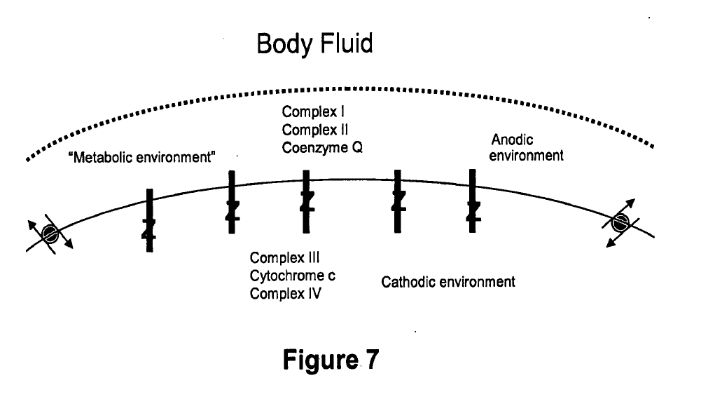

FIG. 7 represents an embodiment of the invention in which the anodic environment contains metabolic enzymes to form a “metabolic environment.” In this case, the environment is a metabolic/anodic environment and comprises the metabolic enzymes (Complex I, Complex II, Coenzyme Q and any oxidoreductases, transferases, hydrolases, lyases, isomerases and ligases useful in metabolizing the oxidizable biological material from the bodily fluid) in contact with the bodily fluid (dotted line indicates access to bodily fluid) such that metabolism at the metabolic/anodic environment occurs constitutively. The cathodic environment comprises Complex III, cytochrome c and Complex IV to mediate the reduction portion. Connectors with resistors run between the metabolic/anodic environment and the cathodic environment, and salt bridges are provided to allow the flow of small ions to complete the circuit.

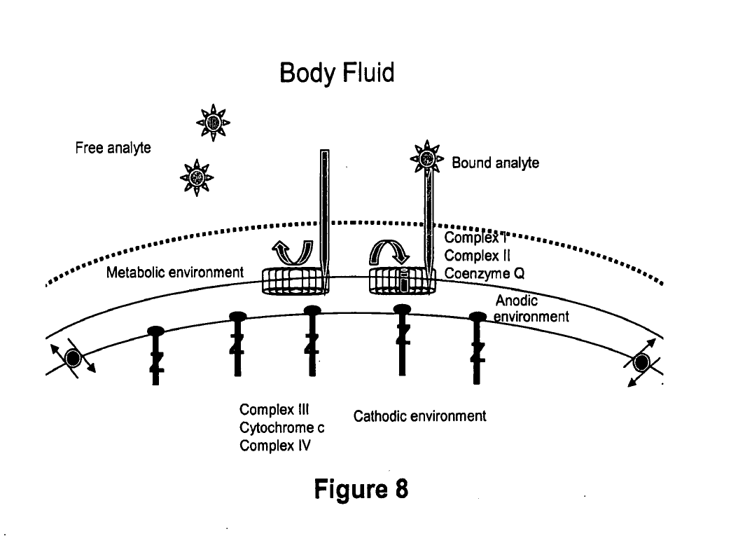

FIG. 8 represents an embodiment in which the metabolic environment is separated from the anodic environment by an impermeable layer which contains gated channels operably linked to receptors for an analyte of interest. In this embodiment, free analytes in the bloodstream come into contact with the receptor and bind to the receptor. When no analyte is bound (left receptor), the channel is closed and no electrons of metabolism in the metabolic environment are able to flow into the anodic environment. When the analyte binds the receptor, the channel opens (right channel), allowing electrons to flow into the anodic environment, and down the connectors to the cathodic environment. Salt bridges allow flow of small ions to complete the circuit.

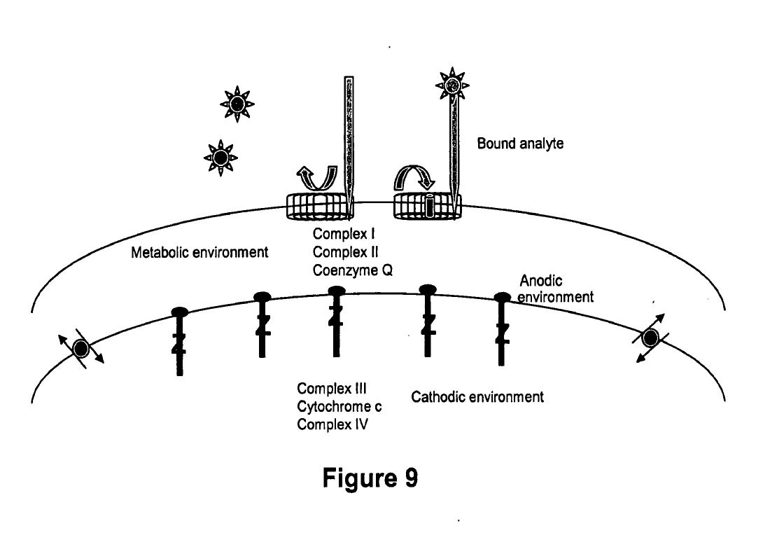

FIG. 9 represents an embodiment of the invention in which the anodic environment contains metabolic enzymes to form a “metabolic environment.” In this case, the environment is a metabolic/anodic environment and comprises the metabolic enzymes (Complex I, Complex II, Coenzyme Q and any oxidoreductases, transferases, hydrolases, lyases, isomerases and ligases useful in metabolizing the oxidizable biological material from the bodily fluid) is sequestered from the bodily fluid by an impermeable barrier (solid line indicates no access to bodily fluid) such that metabolism at the metabolic/anodic environnent does not occur constitutively. The impermeable barrier contains gated channels operably linked to receptors for an analyte of interest. In this embodiment, free analytes in the bloodstream come into contact with the receptor and bind to the receptor. When no analyte is bound (left receptor), the channel is closed and no oxidizable biological material is able to flow into the metabolic/anodic environment. When the analyte binds the receptor, the channel opens (right channel), allowing oxidizable biological material to flow into the metabolic/anodic environment, and electrons generated flow down the connectors to the cathodic enviromnent. Salt bridges allow flow of small ions to complete the circuit.

Although certain presently preferred embodiments of the invention have been specifically described herein, it will be apparent to those skilled in the art to which the invention pertains that variations and modifications of the various embodiments shown and described herein may be made without departing from the spirit and scope of the invention. The examples provided are merely illustrative of the invention and should not be construed as limiting.

### Examples

### Example 1

In an embodiment of the invention, the shell contains a non-magnetic cathode (such as silver) in an aqueous ammonia solution. A non-magnetic wire connects the cathode to an anode outside of the shell (to prevent short-circuiting of the cell and abolition of current generation). The summed electric potential of the reaction (E^{o}=0.323 V) represents the overall thermodynamic favorability of the reaction of the oxidation of glucose and the reduction of Ag⁺ from the aqueous dianuninesilver complex [Ag(NH₃)₂⁺] in solution to elemental silver (pure silver metal).

The oxidization of glucose is a highly thermodynamically favorable reaction because of the high potential chemical energy stored in carbohydrates (it is precisely because of this that glucose is the body's primary means of generating energy for life). It is through the voltaic mechanism described above that the chemical energy is converted to electric energy. Thus, as blood flows around (or through, in the case of tubular or mesh shapes) the device, glucose in the blood passing by the anode is oxidized to gluconic acid (i.e., electrons are stripped from glucose) resulting in the reduction of the silver. Electrons thus flow, from the glucose molecules, across the connector connecting the anode and the cathode, where Ag⁺ is reduced and plated onto the cathode. The device needs only exposure to blood, or other body fluid (such as urine, interstitial fluid, peritoneal fluid etc.), which can be a capillary, for example. It does not need to be implanted in a vein or an artery.

Ions, such as protons (H⁺ ions) are allowed to flow through a semi-permeable membrane in the shell, which serves as a salt bridge to complete the circuit, or through salt bridges engineered into the cell. In this environment, oxygen will tend to diffuse across the salt pore to reoxidize the elemental silver reduced in the redox reaction which will return to solution as Ag⁺/[Ag(NH₃)₂⁺],and allow the circuit to run perpetually. This does not represent a perpetual energy device; the energy for this recharging of the system derives from the high energy of the glucose oxidation reaction (from glucose readily available in the bloodstream, particularly in diabetes) and the thermodynamically favorable oxidation of silver by elemental oxygen. The thermodynamically favorable oxidation of silver by elemental oxygen is why silver naturally develops a patina over time.

The signal may be transmitted in a variety of ways. For example, the increase in current generated in the voltaic cell from increased oxidation of glucose due to increased serum glucose concentration is detected by an external amperemeter (current detector), which is calibrated to interpret incremental increase in current as specific incremental increase in serum glucose concentration (FIG. 1). In this case, the current generated by the glucose meter device (effectively, a bio-voltaic or living-system integrated battery device, by design), can be measured by an external amperemeter. This would measure magnetic flux based on the change of current strength in the implant, just under the skin. The strength of the magnetic field, and thus the magnitude of the magnetic flux, varies proportionately to the magnitude of the current (current is defined as moving charges; moving charges set up magnetic field; a change in magnetic field can be measured over a distance by a change in magnetic flux, which is the basis of common magnetometers).

Alternatively, a micro-radio is built into the voltaic cell and powered by the circuit generated from the oxidation of glucose (FIG. 2). This radio emits higher frequencies radio waves (“higher frequency radio waves” are higher energy waves; this energy increase will come from the higher current (from increasing available electric potential energy derived from increasing glucose concentration, as glucose is a high energy molecule). The higher frequency radio waves are emitted as the current increases secondary to increased serum glucose concentration and thus, increased redox activity. These waves are detected by an external radio receiver, calibrated to translate increases in radio frequency into information on the incremental increases in serum glucose concentration being measured by the implant. This can be achieved using basic, solid-state semiconductor physics. For example, an analogue micro-transistor can be used to stabilize and amplify radio frequency derived from an alternating electric current generated by the device (based on the oxidation of glucose). A radio signal is emitted when a wire carries an alternating current. A simple transmitter results from connecting this system to an antenna. Radio receivers which can be used to receive a radio transmission and be adapted to calculate analyte levels and provide an output are known in the art. An example is found in US 2005/02457299, U.S. Pat. No. 6,585,644, which are incorporated herein by reference.

Harmonic frequency filtration can be achieved through combinations of inductors and capacitors. A bipolar junction transistor is applicable as measurement of current is desired. Otherwise, a field effect transistor, to measure voltage, high gain or digital transistor could be employed, as well. Other types of transistors (dual gates, transistor arrays and combinations, MOSFET, IGBT, IGFET, etc.) of various semiconductor materials (compound, alloys, etc.) may also be used.

In some embodiments, the transistor is part of an integrated circuit, in others it is desirable to use an individual transistor of the device, where the current generated from the oxidation of blood (serum) glucose at the anode flows from the anode to the input terminal of the transistor and modulates the conductivity between the other two terminals, thereby modulating the flow of current between these terminals, allowing for amplification and translation of current changes into radio frequency. In the example of a general transistor, the greater the current (in the case of the glucometer i nplant, progressively higher blood glucose external to the device would generate a progressively higher continuous current) the greater the conductivity between the terminals, and thus the translatability of current into radio frequency, which can be measured by an external radio frequency receiver, a widely available commodity, for display as precise moment to moment glucose level and allow for calculation by this external device of the appropriate insulin dose, once calibrated to the patient.

A digital transistor would allow for the device to act as a microprocessor, which could further process the information on glucose-mediated current generation into various forms of data, within the device, and allow for transmission of these digital data to the external device. Alternatively, a microprocessor may be part of the external device. In an alternative embodiment, microprocessors are included in both the implanted device and the external device.

In cases using detection of voltage, or telemetric detection such as through radio frequency or other wireless communication, an external receiver is calibrated to interpret increases in blood glucose. From this, a receiver (e.g., amperemeter, radio receiver) could be programmed to derive, by common and simple clinical algorithms, the appropriate insulin dose for the attendant serum glucose concentration. Because of slight physiological and metabolic differences between patients, the receiver will be calibrated at time of installation, and its accuracy can be monitored instantaneously by checking against glucometer readings done by finger stick at any time, representing an important safety measure.

The device will be installed by a clinician, similar to installation of depo birth control devices, inconspicuously under the skin of the arm or abdomen. When the patient wishes to check his or her blood sugar, he or she will hold the receiver over the implant, which will read out the implant's electric current or radio frequency at that moment as a glucose level.

### Example 2

A voltaic cell glucometer of the invention is subcutaneously implanted into a diabetic patient who is able to monitor and regulate his or her own insulin levels. The glucometer emits radiowaves which continuously monitor the patient's glucose levels. The signal is received by a radio receiver worn as a wristwatch-type receiver that displays the patient's blood glucose level. The wristwatch receiver also displays the time of day and is equipped with an alarm to alert the patient when his or her glucose level drops to a point at which the patient becomes hypoglycemic, or when an insulin does is required to treat or prevent hyperglycemia.

### Example 3

A voltaic cell glucometer of the invention is subcutaneously implanted into a diabetic patient who is unable to monitor and regulate his or her own insulin levels. The patient is an incapacitated person in the hospital. The glucometer emits radiowaves which continuously monitor the patient's glucose levels. The signal is received by a radio receiver installed near the patient's hospital bed (or alternatively worn in close proximity to the implanted device). The signals emitted by the implanted glucometer are received by the external receiver which continuously calculates and displays the patient's blood glucose level. In addition the receiver further transmits the data by wireless communication to a computer at the nurse's station. The computer at the nurse's station displays the patient's information, vital signs and blood glucose level. The computer may provide an alert if the patient becomes hypoglycemic, or hyperglycemic, or indicate when insulin is required. In addition, the radio receiver on or near the patient may also be equipped with an alarm to indicate hypoglycemia or hyperglycemia. In this way the nurse or medical staff can attend to a diabetic patient in an incapacitated state without the need to draw blood and test glucose levels and thereby maintain better control of a patient's blood glucose level more conveniently and efficiently.

### Example 3

A voltaic cell having cholesterol oxidase complexed to the surface of the anode of the voltaic cell is implanted into a patient having severe hypercholesterolemnia. The voltaic cell is in contact with the patient's blood and cholesterol flowing past the anodes of the cell is oxidized and the electrons flow through the connector to the cathode within the shell of the voltaic cell. The current generated is converted to high frequency radio waves which are received by an external radio receiver which is programmed and calibrated to calculate and display the patient's blood cholesterol level. The patient's cholesterol level is monitored to assess the patient's response to dietary restrictions and therapy with cholesterol-lowering drugs, to help prevent heart attacks and strokes.

### Example 4

A diabetic dog has a voltaic glucometer implanted under the skin of the scruff of the neck. The voltaic cell glucometer detects the dog's blood glucose level which generates current in the voltaic cell. The current is detected by an amperemeter which is worn as a dog collar. The amperemeter is equipped with an alarm that alerts the dog's owner when the dog becomes hypoglycemic or hyperglycemic and displays the blood glucose level. The dog's owner is then alerted to feed the dog or call the veterinarian in hypoglycemia, or administer insulin to the dog in hyperglycemia.

The specific embodiments described herein contain descriptions which are generally applicable to other embodiments of the invention and are not limited solely to the specific embodiment described.

What is claimed is:

1. A voltaic cell for use in the body of a subject comprising:
a biologically inert shell defining an inner compartment, said inner compartment containing an ionic solution that mediates a reduction reaction, said shell having an inner surface and an outer surface, said inner surface being in contact with said ionic solution;
a cathodic environment within said shell in contact with said ionic solution;
an anodic environment attached to said outer surface of said shell, in contact with bodily fluid wherein said anode accepts electrons from an oxidation reaction with at least one oxidizable biological material in said bodily fluid;
a connector connecting said anodic environment and said cathodic environment;
a component that provides resistance disposed along said connector between said anode and said cathode; and
at least one salt bridge disposed within said shell permitting passage of ions between said inner compartment and said bodily fluid.

2. The voltaic cell of claim 1 wherein said cathodic environment comprises at least one cathode.

3. The voltaic cell of claim 1 wherein said anodic environment comprises at least one anode.

4. The voltaic cell of claim 2 wherein said anodic environment comprises at least one anode.

5. The voltaic cell of claim 1 wherein said oxidizable biological material is selected from the group consisting of carbohydrates, cholesterols, fatty acids, amino acids, polypeptides, lipids, hormones and polynucleotides.

6. The voltaic cell of claim 5 wherein said carbohydrate is glucose.

7. The voltaic cell of claim 5 wherein said hormone is selected from the group consisting of estrogen, progesterone, testosterone, growth hormone and thyroid hormone.

8. The voltaic cell of claim S wherein said polypeptide is prostate-specific antigen or a cancer-specific polypeptide.

9. The voltaic cell of claim 1 wherein said ionic solution comprises diamminesilver complex [Ag(NH₃)₂]⁺, silver, copper, or iron salts

10. The voltaic cell of claim 1 wherein said voltaic cell comprises a plurality of cathodes.

11. The voltaic cell of claim 1 wherein said voltaic cell comprises a plurality of anodes.

12. The voltaic cell of claim 11 wherein said voltaic cell comprises a plurality of cathodes.

13. The voltaic cell of claim 1 wherein said shell comprises a plurality of salt bridges.

14. The voltaic cell-of claim 1 wherein said connector is a wire.

15. The voltaic cell of claim 1 wherein said cathode is a non-magnetic metal.

16. The voltaic cell of claim 2 wherein said cathode is selected from the group consisting of silver, aluminum, lead, magnesium, platinum, gold, tin oxide, titanium dioxide, tungsten, non-magnetic alloys, semiconductors, semimetals and organometallic complexes.

17. The voltaic cell of claim 1 wherein said shell is formed from an impermeable material.

18. The voltaic cell of claim 1 wherein said shell is formed from a semi-permeable material.

19. The voltaic cell of claim 1 further comprising an inert barrier having selective permeability to exclude bio-fouling molecules wherein said barrier covers said anode.

20. The voltaic cell of claim 19 wherein said barrier comprises a layer that covers said outer surface of said shell.

21. The voltaic cell of claim 1 wherein said component that provides resistance is selected from the group consisting of a microradio or microprocessor.

22. The voltaic cell of claim 3 wherein said anode is complexed with ammonia (NH₃) molecules.

23. The voltaic cell of claim 3 wherein said anode comprises an enzyme that oxidizes said oxidizable biological material.

24. The voltaic cell of claim 18 wherein said enzyme is selected from the group consisting of glucose oxidase, cholesterol oxidase, hexose oxidase, 1-amino acid oxidase, D-amino acid oxidase, uricase, lactate oxidase, choline oxidase, alcohol oxidase, uricase, xanthine oxidase, bilirubin oxidase, glutamate oxidase, and polyamine oxidase.

25. The voltaic cell of claim 1 further comprising a metabolic environment disposed on the outer surface of said shell or layered over said anodic environment wherein said metabolic environment comprises a plurality of metabolic enzymes and cofactors that oxidize a plurality of oxidizable biological materials.

26. The voltaic cell of claim 25 wherein said metabolic environment comprises metabolic enzymes and cofactors selected from the group consisting of oxidoreductases, transferases, hydrolases, lyases, isomerases, ligases, coenzymes, cofactors, and prosthetic groups.

27. The voltaic cell of claim 26 wherein said plurality of metabolic enzymes and cofactors comprise oxidases, reductases, dehydrogenases, synthases, citric acid cycle enzymes, NAD⁺/NADH, FMN/FMNH₂, NADH dehydrogenase, FAD/IFADH₂, fumarate/succinate, succinate dehydrogenase, acyl CoA dehydrogenase, ubiquinone.

28. The voltaic cell of claim 27 wherein said cathodic environment comprises cytochromes b, c, and a+a₃, iron-containing porphyrin, and ubiquinone.

29. The voltaic cell of claim 25 wherein a single layer comprises said metabolic environment and said anodic environment.

30. The voltaic cell of claim 25 wherein said metabolic environment is disposed in a layer on an outer portion of said anodic environment and is in contact with said bodily fluid, wherein said anodic environment is separated from said metabolic environment by an impermeable boundary layer, wherein said boundary layer comprises gated channels that are operably connected to receptors for an analyte, said receptors extending from said channels to said bodily fluid, wherein binding of said analyte to said receptor causes said channels to open and permit flow electrons to anodic environment.

31. The voltaic cell of claim 28 wherein said anodic environment comprises a impermeable boundary layer between said anodic environment and said bodily fluid, wherein said boundary layer comprises gated channels that are operably connected to receptors for an analyte, said receptors extending from said channels to said bodily fluid, wherein binding of said analyte to said receptor causes said channels to open and permit flow of said oxidizable biological material into said anodic environment.

32. An implantable glucometer comprising a first component and a second component, said first component comprising:
a biologically inert, shell defining an inner compartment, said inner compartment containing an ionic solution that mediates a reduction reaction, said shell having an inner surface and an outer surface, said inner surface being in contact with said ionic solution;
a cathodic environment within said shell in contact with said ionic solution;
an anodic environment on said outer surface of said shell, in contact with bodily fluid wherein said anodic environment accepts electrons from an oxidation reaction of glucose in said bodily fluid;
a connector connecting said anodic environment and said cathodic environment;
a component that provides resistance disposed along said connector between said anodic environment and said cathodic environment; and
at least one salt bridge disposed within said shell connecting said inner compartment and an outside portion of said shell;
said second component comprising a detection device, wherein said detection device detects a signal from said first device and provides a value that correlates with blood glucose level in said subject.

33. The glucometer of claim 32 wherein said cathodic environment comprises at least one cathode.

34. The glucometer of claim 32 wherein said anodic environment comprises at least one anode.

35. The glucometer of claim 34 wherein said cathodic environment comprises at least one cathode.

36. The glucometer of claim 32 wherein said second component is an amperemeter.

37. The glucometer of claim 32 wherein said component that provides resistance is a microradio or microprocessor.

38. The glucometer of claim 32 further comprising an inert barrier layer having selective permeability for small ions covering said anode.

39. The glucometer of claim 32 wherein said anodic environment comprises glucose oxidase.

40. The glucometer of claim 39 wherein said glucose oxidase is complexed with at least one anode.

41. The glucometer of claim 32 wherein said anodic environment comprises ammonia.

42. The glucometer of claim 41 wherein said ammonia is complexed with at least one anode.

43. The glucometer of claim 32 wherein said ionic solution comprises diamminesilver complex [Ag(NH₃)₂]⁺, silver, copper or iron salts.

44. The glucometer of claim 32 wherein said shell comprises a plurality of salt bridges.

45. The glucometer of claim 32 wherein said connector is a wire.

46. The glucometer of claim 33 wherein said cathode is a non-magnetic metal.

47. The glucometer of claim 33 wherein said cathode is selected from the group consisting of silver, aluminum, lead, magnesium, platinum, gold, tin oxide, titanium dioxide, tungsten, non-magnetic alloys, semiconductors, semimetals and organometallic complexes.

48. The glucometer of claim 32 wherein said shell is formed from an impermeable material.

49. The glucometer of claim 32 wherein said shell is formed from a semi-permeable material.

50. The glucometer of claim 32 further comprising an inert barrier having selective permeability to exclude bio-fouling molecules wherein said barrier covers said anode.

51. The glucometer of claim 50 wherein said barrier comprises a layer that covers said outer surface of said shell.

52. A method of measuring blood glucose comprising:
implanting a first device in the body of a subject such that said first device is in contact with blood;
allowing said first device to contact glucose in said blood whereby oxidation of glucose contacting said first device initiates an electric current in said first device; and
detecting said current with a second device;
wherein said first device comprises a glucometer of claim 32; and
wherein said second device detects current and provides an indication of blood glucose level in said subject.

53. The method of claim 52 wherein said second device is an amperemeter.

54. The method of claim 52 wherein said component that provides resistance is a radio emitter and said second device is a radio receiver.

55. A method of powering an implantable medical device comprising connecting said device to at least on voltaic cell of claim 1.

56. The method of claim 55 wherein a plurality of voltaic cells is provided in parallel.

57. A voltaic cell for use in the body of a subject comprising:
a biologically inert shell defining an inner compartment, said inner compartment containing a solution that mediates an oxidation reaction, said shell having an inner surface and an outer surface, said inner surface being in contact with said solution;
an anodic environment within said shell wherein said anodic environment comprises electron donors, antioxidants or active reductants;
a cathodic environment on said outer surface of said shell, in contact with bodily fluid;
a connector connecting said anodic environment and said cathodic environment;
a component that provides resistance disposed along said connector between said anode and said cathode; and
at least one salt bridge disposed within said shell permitting passage of ions between said inner compartment and said bodily fluid.

58. The voltaic cell of claim 57 wherein said electron donors comprise NADH, FADH₂, and FMNH₂.

59. The voltaic cell of claim 57 wherein said antioxidants comprise glutathione or ascorbate.

60. The voltaic cell of claim 57 wherein said active reductants comprise sodium borohydride.
